(19) United States
(12) Patent Application Publication
(10) Pub. No.: US 2026/0334934 A1
(43) Pub. Date: Oct. 29, 2026

(54) TRACTION DEVICES, SYSTEMS, AND METHODS

(71) Applicant: Boston Scientific Medical Device Limited, Galway (IE)

(72) Inventors: Nachiket Vilas Gole, Pune (IN); Deepak Kumar Sharma, Muzaffarnagar (IN); Rajivkumar Singh, Pune (IN); Sharath Kumar G, Kanakapura (IN); Shrikant Vasant Raut, Mumbai (IN)

(73) Assignee: Boston Scientific Medical Device Limited, Galway (IE)

(21) Appl. No.: 19/660,960

(22) Filed: Apr. 28, 2026

Related U.S. Application Data
(60) Provisional application No. 63/796,563, filed on Apr. 29, 2025.

(51) Int. Cl. A61B 17/02; A61B 17/00

(52) U.S. Cl. CPC A61B 17/0218; A61B 2017/00867; A61B 2017/0225

(57) ABSTRACT

A tissue traction system with a tissue traction device operably separably coupled with a delivery device. The tissue traction device may be engaged with tissue and separated from the delivery device by breaking a frangible connection between the tissue traction device and the delivery device. The tissue traction device may be implanted into tissue and may have a shape memory configuration which is constrained by the tissue upon implanting of the tissue traction device with respect to the tissue, and which is allowed to relax into an unconstrained curved configuration as the target tissue is cut from surrounding to tissue to apply traction to the cut target tissue.

### CROSS-REFERENCE TO RELATED APPLICATIONS

This application claims the benefit of priority under 35 U.S.C. § 119 to U.S. Provisional Application No. 63/796,563, filed on Apr. 29, 2025, the entire disclosure of which is hereby incorporated by reference herein for all purposes.

### FIELD

The present disclosure relates generally to the field of medical devices, systems, and methods for applying traction to anatomical tissue.

### BACKGROUND

Endoscopic Mucosal Resection (EMR) is an endoscopic procedure which involves the placement of a snare around tissue and either contracting the snare to transect (cold), or using electrosurgical current to transect (hot) the encircled tissue. Endoscopic Submucosal Dissection (ESD) is an endoscopic procedure, first developed in Japan in the late 1990's, used to remove superficial gastrointestinal neoplasms via en bloc resection as a reaction to the shortcomings of EMR. Typically, ESD procedures are technically more challenging than EMR procedures, with longer procedural times and higher rates of complications. However, en bloc resection allows good histopathological assessment of lesion margins, lower recurrence rates, and a greater minimally invasive potential for cure.

Minimally-invasive (e.g., transluminal, transcatheter, endoscopic, etc.) surgical techniques like ESD and EMR typically allow for faster recovery than with open or laparoscopic surgical procedures. However, because such procedures are minimally invasive, there is limited space to maneuver within the body, and such procedures typically require a high degree of expertise. In procedures involving cutting of tissue, one of the largest time and complexity drivers is managing the tissue being cut. The loose section of tissue may obstruct visibility, such as by falling on the instruments being used, occluding visibility of the imaging device (e.g., camera), and creating a hindrance affecting movement of the instruments used during the procedure and in reaching all regions and depths of the target tissue being cut. Various solutions for lifting the cut (and often hanging) mass of tissue, thus clearing the path for visibility and operation of medical tools and devices, have been developed. However, positioning of the elements used with such solutions may be challenging, particularly in a space-restricted environment. Moreover, as tissue is cut, a continual increase in traction is required, presenting various challenges during the procedure. Additionally, the devices used with such solutions may require separate medical tools than those used to perform the procedure, and such tools may even require a separate working channel, thereby potentially increasing the size and/or complexity of the delivery system. As endoscopic submucosal dissection continues to evolve with new techniques, and new instruments are designed for making the procedure faster and safer, there remains a need for improvements to application of traction to tissue during the ESD procedure. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In some aspects, an implantable tissue traction device formed in accordance with various principles of the present disclosure includes an elongated biocompatible shape memory material defining a distal end and a proximal end of said tissue traction device, and in which the distal end is formed as a sharp pointed end capable of penetrating into target tissue within a patient's body; and the shape memory material is set into a curved configuration constrained by the target tissue into a substantially straight configuration and releasable into a relaxed curved configuration upon cutting of the target tissue from surrounding tissue.

In some aspects, the proximal end of the tissue traction device is tapered.

In some aspects, the radius of curvature of the tissue traction device varies along the length thereof.

In some aspects, the tissue traction device has a middle region with a radius of curvature larger than a radius of curvature of either of the proximal end or the distal end.

In some aspects, the tissue traction device has a curved middle region, and the proximal end and the distal end extend obliquely with respect to the middle region.

In some aspects, the tissue traction device has a thickness which varies along the length of the tissue traction device.

In some aspects, the proximal end is separably coupled to a delivery device. In some aspects, a weakened area is defined between the tissue traction device and the delivery device to facilitate separation of the traction device from the delivery device to deploy the tissue traction device with respect to the target tissue. In some aspects, the tissue traction device is formed as a distal portion of an elongate element forming the delivery device. In some aspects, the tissue traction device is separately formed from and operably separably coupled to a distal end of the delivery device.

In some aspects, a tissue traction system formed in accordance with various principles of the present disclosure includes a tissue traction device formed of an elongated biocompatible shape memory material defining a distal end and a proximal end of the tissue traction device; a delivery device separably coupled with the proximal end of the tissue traction device; and a delivery sheath defining a lumen through which the tissue traction device and the delivery device are advanceable for delivery to a target tissue within a patient. In some aspects, the tissue traction device is constrained in a constrained substantially straight configuration within the lumen of the delivery sheath and relaxes to a relaxed curved configuration when extended distally out of the delivery sheath.

In some aspects, the tissue traction device and the delivery device are formed from the same monolithic elongate element.

In some aspects, a weakened area is defined between the tissue traction device and the delivery device to facilitate separation and deployment of the tissue traction device with respect to the target tissue.

In some aspects, the tissue traction device and the delivery device are formed of a wire and the weakened area is a cut in the wire.

In some aspects, only the distal portion of the monolithic elongate element is shape set to form the tissue traction device.

In some aspects, the tissue traction device and the delivery device are separately formed and separably coupled together. In some aspects, the tissue traction device and the delivery device are coupled together via a frangible connection.

In some aspects, a method of applying traction to a target tissue in accordance with various principles of the present disclosure includes advancing a delivery device to the target tissue, the delivery device having a tissue traction device with a proximal end separably operably coupled to a distal end of the delivery device; advancing a distal end of the tissue traction device into the target tissue, the tissue traction device being formed of a shape memory material shape set into a curved configuration which is constrained by the target tissue into a constrained configuration; separating the proximal end of the tissue traction device from the distal end of the delivery device; and cutting the target tissue with respect to surrounding tissue, thereby releasing the constraining force of the target tissue on the tissue traction device to allow the tissue traction device to return to a relaxed curved configuration lifting the target tissue as it is being cut from surrounding tissue.

In some aspects, the method further includes advancing the tissue traction device and the delivery device to the target tissue within a delivery sheath which constrains the tissue traction device into a constrained configuration.

In some aspects, the method further includes breaking a connection between the tissue traction device and the delivery device to separate the tissue traction device from the delivery device to deploy the tissue traction device with respect to the target tissue.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1A illustrates a perspective view of an example of an embodiment of a tissue traction system formed in accordance with aspects of the present disclosure with a tissue traction device in a delivery configuration constrained within a delivery sheath.

FIG. 1B is a view similar to that of FIG. 1A, but with the tissue traction device advanced out of the delivery sheath and in a generally unconstrained configuration.

FIG. 2 is an elevational view of an example of an embodiment of a tissue traction device such as illustrated in FIG. 1A and FIG. 1B.

FIG. 2A is a detail view of FIG. 2.

FIG. 3A is a longitudinal cross-sectional view along line IIIA-IIIA of the tissue traction device illustrated in FIG. 2.

FIG. 3B is a cross-sectional view of another example of an embodiment of a tissue traction device formed in accordance with various principles of the present disclosure.

FIG. 3C is a cross-sectional view of another example of an embodiment of a tissue traction device formed in accordance with various principles of the present disclosure.

FIG. 3D is a cross-sectional view of another example of an embodiment of a tissue traction device formed in accordance with various principles of the present disclosure.

FIG. 4 is an elevational view of another example of an embodiment of a tissue traction device formed in accordance with various principles of the present disclosure.

FIG. 4A is a perspective view of a section of a tissue traction device such as illustrated in FIG. 4.

FIGS. 5A, 5B, 5C, and 5D illustrate an example of use of a tissue traction system such as illustrated in FIG. 1.

FIGS. 6A-6B illustrate an example of a use of a tissue traction system such as illustrated in FIG. 1 to deploy more than one tissue traction device.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

There has been a growing interest in the medical field in minimally-invasive procedures, e.g., transcatheter, endoscopic, laparoscopic, etc., which do not require open surgery (cutting open the patient), but, instead, access a target site within the patient via a natural orifice (or, in some instances, a small incision not considered to constitute an open-surgery cut). For the sake of convenience, and without intent to limit, such procedures may be broadly referenced herein as transluminal procedures, in contrast with surgical procedures utilizing large surgical openings made in the patient for generally direct access (e.g., without navigating through a device or internal body passage) to the site at which a procedure is to be performed (e.g., target tissue). Various endoluminal surgical (ELS) procedures have been developed, such as, without limitation, Endoscopic Mucosal Resection (EMR), Endoscopic Submucosal Dissection (ESD), to be performed within a patient and not in direct view of the medical professionals performing the procedure. The lack of direct visibility, as well as typical limited space for maneuvering of medical instruments presents various challenges to the medical professionals performing the procedure.

Although EMR can be performed in piecemeal technique, pathology on tissue fragments is often challenging and patients require more frequent follow-up endoscopies due to higher rate of incomplete resections and recurrences. While EMR is associated with relatively low technical complexity, shorter procedure times, and a low risk of adverse events, en bloc EMR is limited typically to lesions which are less than 2.0 cm in diameter/width. More specifically, for large sessile or laterally spreading polyps, en bloc resection with convention EMR is nearly impossible for lesions greater than 2 cm. For colorectal lesions of greater than 2 cm, en bloc resection can only be achieved in about 30% of cases.

Medical professionals thus often turn to ESD for resection of challenging lesions, as well as large lesions. It is not uncommon for ESD to be utilized to remove neoplasms between 2-7 cm. The European Society of Gastrointestinal Endoscopy (ESGE) recommends ESD be used first line in the removal of Gastric Superficial neoplastic lesions with very low risk of lymph node metastases (EMR may be considered if the lesion is less than 10 to 15 mm with a very low probability of advanced histology). Often, ESD is also recommended first line for superficial esophageal squamous cell cancer (with EMR an option for lesions which are less than 10 mm). For colonic and rectal lesions, ESGE recommends removal of lesions with polypectomy or EMR. However, ESD can be considered if there is a high suspicion of limited submucosal invasion (indicated by depressed morphology and an irregular or non-granular surface pattern), and especially if the lesion is greater than 20 mm, or in lesions that otherwise cannot be optimally and radically removed by snare-based techniques (such as EMR techniques).

Although ESD has been in practice for two decades, ESD remains a very technically challenging procedure. In Japan, where ESD procedure is best established, the complication rates (including perforation, peritonitis, and bleeding) are 3.5% for gastric ESD, 3.3% for esophageal ESD, and 4.6% for Colorectal ESD. Outside of Japan and China, EDS is practiced by a relatively small number of highly technically-skilled endoscopists and surgeons. Long procedure times and high risk of perforation are still significant challenges which have prevented broad physician adoption.

In accordance with various principles of the present disclosure, devices, systems, and methods are described which may be used in minimally-invasive procedures, such as ESD, to move anatomical tissue (typically a flap of tissue formed by cutting a target tissue with respect to adjacent or surrounding tissue) out of the way of instruments being used to perform a procedure within the patient's body. It will be appreciated that reference may be made herein simply to “tissue” instead of “anatomical tissue” or “anatomical structure” for the sake of convenience and without intent to limit. In some aspects, the present disclosure is directed to devices, systems, and methods for moving tissue, such as to separate and/or lift and/or apply traction to tissue within a patient's body. Traction is a foundational technique utilized in open and laparoscopic surgery. Traction may be used to move tissue or other anatomical structures and may be advantageously used to greatly enhance visualization of anatomy, tissue planes, and potential hazards such as nerves or blood vessels. Additionally or alternatively, traction may be used to impart a tension force to the tissue, which may change the way the tissue behaves as it is being cut or dissected. For instance, traction may be used in surgical procedures to “present” the tissue to be cut to the cutting instrument in an orientation or manner that makes the cutting easier and/or helps to ensure that the cut is made through the desired tissue in the desired direction. In some aspects, traction alters the nature and/or properties of tissue in a manner which may facilitate performance of a procedure with respect to or in the vicinity of the tissue under traction. It will be appreciated that principles of the present disclosure are applicable to tissue which is cut as well as tissue or other anatomical structures which are not cut. For instance, principles of the present disclosure are applicable for providing traction and/or counter-traction during various medical procedures, including, without limitation, surgical procedures that involve cutting of tissue. The use of traction is a basic precept of surgery during cutting procedures, but should be appreciated as having broader applicability as well.

In addition to the procedural benefits of using traction during endoscopic procedures, there is a mounting body of evidence that devices aiding with traction during endoscopic submucosal dissection reduce the technical complexity of the procedure, and significantly lower the learning curve associated with trainees and/or new ESD procedure adopters.

To date, current traction solutions, both commercialized and physician made, do not adequately address true user needs. At best, commercially-available traction solutions leave significant room for improvement with regard to efficiency, effectiveness, flexibility, and ease of use. In open surgery, traction typically is applied using the non-dominant hand directly or through a surgical instrument (typically some kind of forceps). In laparoscopic surgery, traction is applied via tissue graspers introduced through one of the surgical ports, which provide access to the target body cavity or space. This can be done by the lead surgeon, assisting surgeon, or scrub nurse. In endoscopic surgery, however, multiple portals for accessing the target surgical site generally are not provided, and, typically, only the endoscope (or other access device) provides both visualization and access. Although there are some endoscopes with two working channels, or devices that can attach to the outside of the endoscope to provide a secondary working channel, all of these access channels are either integral to the endoscope or run parallel to it, and therefore are directionally constrained, and limited in the degrees of freedom of movement and control that can be achieved when maneuvering other devices to the target site. Moreover, the medical scopes and even auxiliary lumens, and often also the anatomical site of the procedure itself, offer limited space for manipulation of additional instruments, tools, devices, accessories, etc. (such terms being usable interchangeably herein without intent to limit), such as to manipulate and move tissue out of the way of the instrument performing a procedure with respect to the tissue.

In some aspects, a traction device formed in accordance with various principles of the present disclosure is engageable with anatomical tissue of a patient to impart a traction force with respect to the tissue. It will be appreciated that reference may be made, interchangeably and without intent to limit, to a treatment site, anatomical site, delivery site, deployment site, implant/implantation site, site of implantation, target site, etc., at which a tissue traction system of the present disclosure is to be used and/or a procedure is to be performed. In some aspects, target tissue is defined within the treatment site and with respect to which a procedure is to be performed. The target tissue may be, but is not necessarily limited to, a lesion, polyp, biopsy sample area, etc.

In some aspects, a tissue traction device formed in accordance with various principles of the present disclosure is formed of a biocompatible shape memory material. In some aspects, the traction device is implanted within a layer of anatomical tissue of the target tissue. For instance, in some aspects, the traction device is implanted into mucosal tissue to be separated from (e.g., cut, resected, dissected, etc.) submucosal tissue.

In some aspects, a tissue traction device formed in accordance with various principles of the present disclosure is delivered and deployed in a generally straight constrained configuration, but is biased into a curved, relaxed configuration when no constraining forces hold the tissue traction device in a straight configuration. When the tissue traction device is deployed with respect to tissue (engaged with, implanted into, etc.), the tissue traction device may be in a generally constrained configuration, held in place with respect to the target tissue. As the target tissue is cut away from surrounding and/or underlying tissue, the constraining force on the tissue traction device is relieved, and the inherent bias of the tissue traction device shifts the tissue traction device to a curved configuration, lifting the target tissue with which the tissue traction device is engaged. The tissue traction device thus lifts the target tissue away from surrounding and/or underlying tissue to remove the flap of tissue being created by cutting the target tissue to allow the medical professional to view the target tissue and the surrounding anatomical tissue without interference of the flap of tissue.

Additionally or alternatively, in some aspects, a tissue traction device formed in accordance with various principles of the present disclosure is deliverable operably coupled to a distal end of a delivery device. In some aspects, the delivery device is an elongated element, such as a pull/push wire. In some aspects, the tissue traction device is releasably operably coupled with the delivery device to allow separation and deployment of the tissue traction device within the patient's body without the need for a further tool or actuator. For instance, the tissue traction device may be separably coupled to the delivery device, such as via a frangible connection. In some aspects, the tissue traction device may be formed from a distal end of an elongate element, with a frangible area separating the tissue traction device portion from the delivery device portion of the elongate element. In some aspects, the traction device is formed separately from the delivery device and releasably coupled thereto. In some aspects, twisting or other movement of the delivery device, such as after engagement of the tissue traction device with target tissue, facilitates separation of the tissue traction device from the delivery device.

In some aspects, devices, systems, and methods of the present disclosure provide an integrated device and delivery system assisting medical professionals to perform procedures in which tissue is to be lifted without the need for manipulation of instruments alongside the instrument being used to perform a procedure to be assisted by a tissue traction device of the present disclosure, thereby contributing to reducing procedure complexities and procedure times.

Various embodiments of traction devices, systems, and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiments are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features are identified by common reference elements and, for the sake of brevity and convenience, and without intent to limit, the descriptions of the common features are generally not repeated. For purposes of clarity, not all components having the same reference number are numbered. It will be appreciated that, in the following description, elements or components similar among the various illustrated embodiments with reference numbers greater than 100 are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

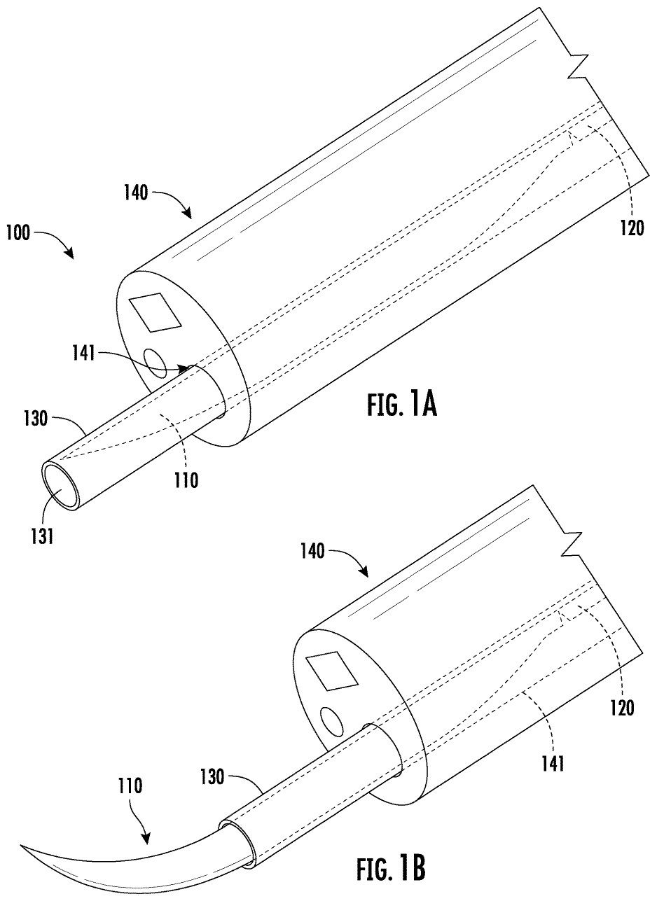

Turning now to the drawings, an example of an embodiment of a tissue traction system 100 formed in accordance with various principles of the present disclosure is illustrated in FIG. 1A and FIG. 1B. The tissue traction system 100 includes a tissue traction device 110 configured to be engaged with target tissue to apply traction to the target tissue, a delivery device 120 operably associated with the tissue traction device 110, and a delivery sheath 130. Optionally, the tissue traction system 100 is delivered through another tubular delivery device 140. In the example of an embodiment illustrated in FIG. 1A and FIG. 1B, the tubular delivery device 140 is a medical scope such an endoscope, and the tissue traction system 100 is deliverable through a working channel 141 of the medical scope. In some aspects, the tubular delivery device 140 is considered to be a part of the tissue traction system 100. In some aspects, the various components of the tissue traction system 100 (tissue traction device 110, delivery device 120, delivery sheath 130, and/or tubular delivery device 140) are sufficiently flexible to be extended into a curved and/or tortuous passage within a patient's body and advanced (e.g., transluminally) to a target tissue within the patient's body. In some aspects, at least the delivery device 120, delivery sheath 130, and/or tubular delivery device 140 are elongated and extend proximally to a proximal end capable of being controlled by a medical professional, such as to move (axially translate, such as advance distally or retract proximally; rotate, etc.) each, such as separately and independently of one another.

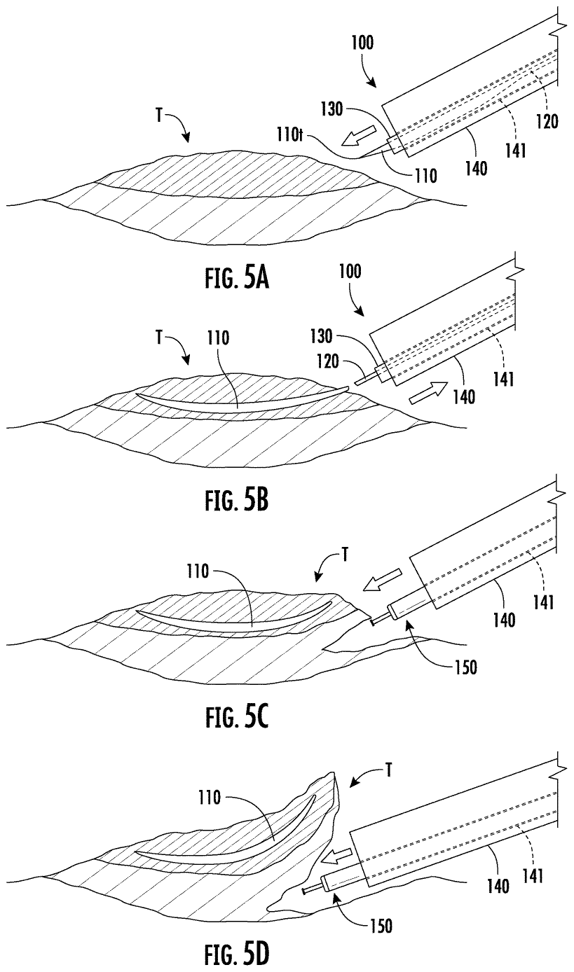

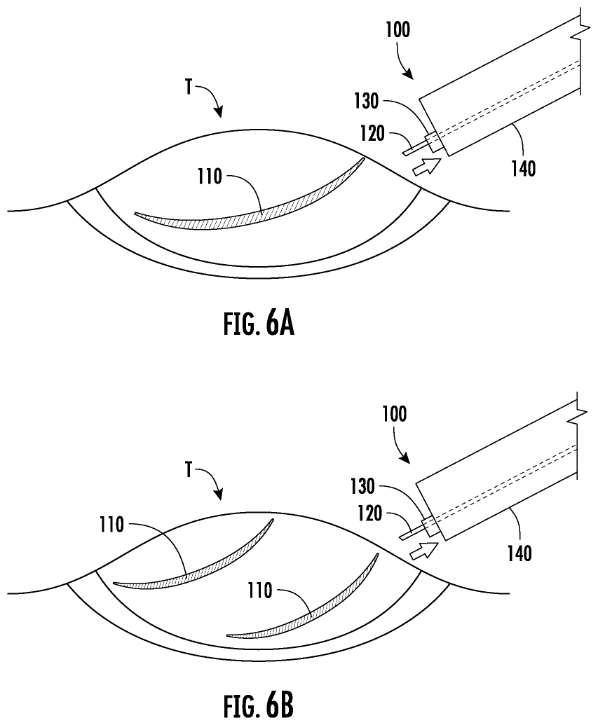

As may be appreciated with reference to (and upon comparison of) FIG. 1A and FIG. 1B, the tissue traction device 110 has a nonlinear/curved configuration (referenced herein as “curved” for the sake of convenience and without intent to limit) when not constrained. The tissue traction device 110 may be constrained within a lumen 131 of the delivery sheath 130, such as into a generally linear or straight configuration (which may be referenced herein as a constrained configuration), as illustrated in FIG. 1A. Likewise, when the tissue traction device 110 is engaged with tissue, such as illustrated in FIGS. 5A-5D, 6A, and 6B and discussed in greater detail below, the tissue which is engaged with the tissue traction device 110 may constrain the tissue traction device 110 to follow and/or conform to configuration of tissue to which the tissue traction device 110 is engaged. Upon cutting around target tissue to which the tissue traction device 110 is engaged, to separate the target tissue from adjacent and/or surrounding tissue, the constraining force (e.g., from adjacent/surrounding tissue maintaining a position and/or configuration) of the target tissue on the tissue traction device 110 is released (typically gradually, such as in conjunction with the cutting of the target tissue), allowing the tissue traction device 110 to shift to an unconstrained curved, flexed, bent, or otherwise nonlinear configuration, thereby lifting the tissue, as described in further detail below.

In some aspects, the tissue traction device 110 is formed of a shape memory material, such as, without limitation, nitinol. In some aspects, the tissue traction device 100 is formed of a material which has a shape memory to some extent, e.g., a flexible material which may spring back to its original form after being deformed. For instance, the tissue traction device 100 may be formed from a material, such as polypropylene, which may be formed into (e.g., molded in) a particular shape (e.g., curved as described herein) and deformed, during use, in its elastic zone to some extent (e.g., may operate in an elastic zone, such as within a range acceptable for use as a tissue traction device 100) so that the tissue traction device 100 can be temporarily deformed and then regain its original shape without permanent deformation. The tissue traction device 110 may thus be formed in, or curved or flexed or bent or otherwise deformed from a straight configuration into, a relaxed curved, flexed, bent, or otherwise nonlinear preset configuration. The curved, flexed, bent or otherwise nonlinear preset configuration is set as a relaxed configuration so that the tissue traction device 110 returns to the such configuration (e.g., after being deformed or otherwise constrained into a “constrained” configuration) to be usable in moving the tissue with which the tissue traction device 110 has been engaged (e.g., lifting or otherwise moving the tissue out of the way of a medical device performing a procedure with respect to a target tissue area (e.g., surrounding or adjacent to the target tissue). In some aspects, the shape (e.g., curved shape) of the tissue traction device 110 can also be adjusted or shaped by the medical professional before or during deployment. In some aspects, the shape memory material of the tissue traction device 110 is selected or treated to be active or activated at temperatures within the patient's body. In some aspects, the austenite finish (Af) temperature of the shape memory material (e.g., alloy) needs to be above the patient's body temperature so that the shape memory is retained during the procedure. In some aspects, the tissue traction device 110 may be considered to be elongated as well as curved, or elongated as well as substantially straight (e.g., not completely straight, but more straight than curved).

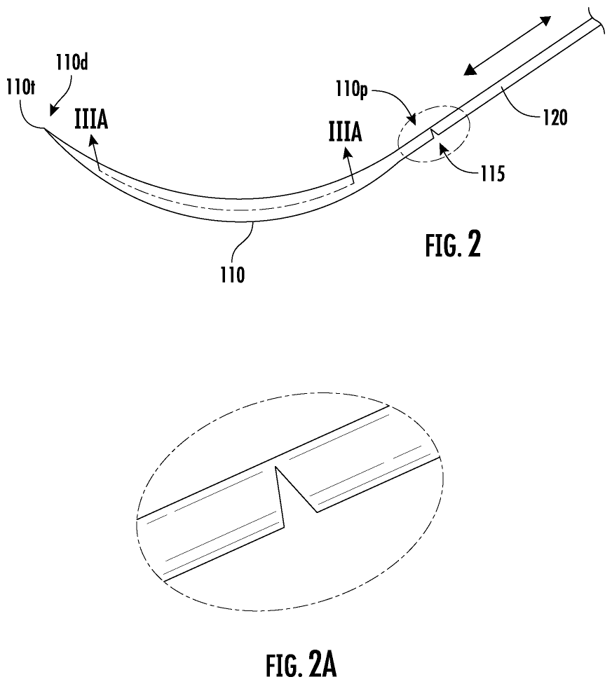

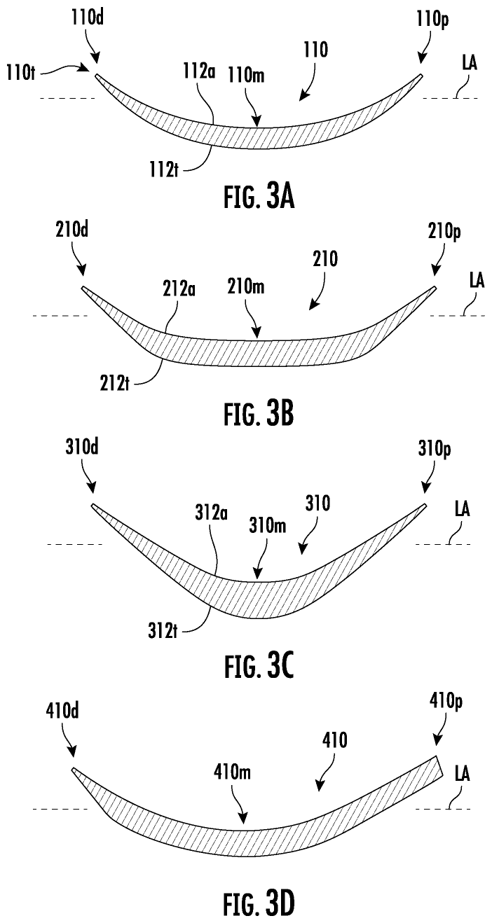

An example of a fully unconstrained configuration of a tissue traction device 110 formed in accordance with various principles of the present disclosure is illustrated in FIG. 2. As may be appreciated, no external constraining forces, such as from a delivery sheath 130 or tissue, are exerted on the illustrated example of an embodiment of a tissue traction device 110, and the tissue traction device 110 is thus in an unconstrained, relaxed configuration. As may be appreciated with reference to FIG. 2 and the longitudinal cross-sectional view along line III-III of FIG. 2, as illustrated in FIG. 3, in the unconstrained configuration, the tissue traction device 110 may have a generally curved configuration. More specifically, the tissue traction device 110 may be described as having opposite surfaces 112t, 112a each having a longitudinal extent generally along a longitudinal axis LA of the tissue traction device (between the distal end 110d to the proximal end 110p), as well as a transverse extent transverse to (e.g., orthogonal to) the longitudinal axis LA (between the lateral sides of the tissue traction device extending along, generally parallel to the longitudinal axis LA). The tissue-engaging surface 112t, which is to be engaged with tissue, is generally convexly curved, whereas the surface 112a facing away from tissue is generally concavely curved. In some aspects, the distal end 110d and/or the proximal end 110p of the tissue traction device 110 are tapered relative to the mid-region 110m. In some aspects, the distal end 110d has a distal tip 110t which may extend from a distalmost end of the tissue traction device 110 and may be pointed, and optionally sufficiently sharp to penetrate or pierce tissue to facilitate deployment/implantation into tissue, such as described in further detail below. The transverse extent (which may be considered a width, generally perpendicular to the longitudinal axis LA) typically is selected to be smaller than the internal diameter of the lumen 131 of the delivery sheath 130. Typically, the outer diameter of the delivery sheath 130 is selected to be extendable through the working channel of a tubular delivery device 140, which may be approximately 2 mm or less (e.g., a typical inner diameter of a working channel of a medical scope such as an endoscope). The implantable length of tissue traction device 110 can be nearly equal to or smaller than the size of the target tissue (e.g., the axial length of a lesion or polyp being treated). In some aspects, the implantable length of the tissue traction device 110 may be up to approximately 5 cm. There can be multiple variants of tissue traction devices 100 formed in accordance with various principles of the present disclosure for selection by the medical professional, such as based on the length of the target tissue with respect to which the tissue traction device 100 is to be used/deployed (e.g., the size of the lesion being treated).

It will be appreciated, however, that various configurations other than the configuration illustrated in FIG. 2 and FIG. 3A are within the scope of the present disclosure. In accordance with various principles of the present disclosure, various properties of a tissue traction device formed in accordance with various principles of the present disclosure (e.g., size, shape, configuration, dimensions, relative proportions, curvature, cross-sectional shape, flexibility, resiliency, etc.) may be selected based on the ultimate deployment site of the tissue traction device 110 and/or the desired traction to be applied by the tissue traction device 110. For instance, the cross-sectional configuration of a tissue traction device formed in accordance with various principles of the present disclosure need not be limited to the configuration illustrated in FIG. 3A, with tapered ends 110d, 110p and a generally continuous curvature. In some aspects, the curvature of the tissue traction device along its longitudinal axis LA need not be continuous. For instance, the curvature towards the ends of a tissue traction device formed in accordance with various principles of the present disclosure may be different from the curvature of a mid-region of the tissue traction device. As referenced herein, the “ends” of the tissue traction device are not limited to the distalmost ends, but include a longitudinal extent from a distalmost end towards a mid-region proportionate to the total length (along the longitudinal axis LA) of the tissue traction device to exert traction against tissue with respect to which the tissue traction device is engaged, in a manner discernible by those of ordinary skill in the art.. In some aspects, an “end” is approximately 10%-40% of the length of the tissue traction device 100. For instance, in the example of an embodiment of a tissue traction device 210 illustrated in FIG. 3B, the mid-region 210m is generally straight or has a noticeably larger radius of curvature than one or both ends 210d, 210p, thereby imparting a generally flattened configuration to the mid-region 210m. One or both of the proximal end 210p or the distal end 210d extends obliquely with respect to the mid-region 210m. The ends 210d, 210p may be generally straight or curved (typically with a smaller radius of curvature than the mid-region 210m). In some aspects, the curvature along the tissue-facing surface 212t of the tissue traction device 210 may be different from the curvature along the surface 212a which is to face away from the tissue.

In the example of an embodiment of a tissue traction device 310 illustrated in FIG. 3C, the illustrated cross-sectional shape along the longitudinal axis LA is generally a gentle V-shape with a curved mid-region 310m and ends 310d, 310p extending away from the tissue-facing surface 312t of the tissue traction device 310 in a direction toward the surface 312a which is to face away from tissue. In some aspects, the curvature along the tissue-facing surface 312t may be different from the curvature along the surface 312a which is to face away from the tissue. In some aspects, the mid-region 310m has the thickest cross-section along the length of the tissue traction device 310, with the thickness of the tissue traction device 310 tapering towards either end 310d, 310p. The ends 310d, 310 p of the tissue traction device 310 may be generally straight or curved (typically with a larger radius of curvature than the mid-region 310m).

It will be appreciated that the cross-sectional shapes of a tissue traction device formed in accordance with various principles of the present disclosure need not be symmetrical. For instance, as noted above, the tapering of the distal end and the proximal end of the tissue traction device need not be to the same extent. In the example of an embodiment of a tissue traction device 410 illustrated in FIG. 3D, the distal end 410d tapers, such as to a pointed (e.g., sharp) distal tip 410t, whereas the proximal end 410p remains thicker. In some aspects, only the distal end 410d is tapered, and the proximal end 410p is substantially the same thickness as the mid-region 410m.

In some aspects, the proximal end 110p of a tissue traction device 110 formed in accordance with various principles of the present disclosure may have substantially the same thickness as the delivery device 120. Returning to FIG. 2, in some aspects, the delivery device 120 is operably coupled to the proximal end 110p of the tissue traction device 110. The thickness of the proximal end 110p of the tissue traction device 110 may be substantially equal to the thickness of the delivery device 120 such as for a smooth transition therebetween.

In some aspects, the tissue traction device 110 and the delivery device 120 are monolithic, formed from single, common element such as a common elongate element such as a push/pull wire. In some aspects, a distal end of an elongate element forming the delivery device 120 is treated (e.g., heat-treated) to have a set curved shape (shape set into a shape such as described above) to define a traction device 110 which is separable from the proximal delivery device 120 portion of the elongate element (e.g., straight or otherwise not shape-set). The distal portion of the elongate element which forms the tissue traction device 110 can be in the form of short, curved segment of the elongate element, such as a few centimeters long, or other length suitable for use at a target area to apply traction to target tissue. In some aspects, the cross-section of the tissue traction device 110 portion of the elongate element is different from the cross-section of the proximal portion of the elongate element which forms the delivery device 120. For instance, the proximal portion of the elongate element forming the delivery device 120 may be rounded or circular (such as for torquability) whereas the distal portion of the elongate element forming the tissue traction device 110 may have a generally flattened (e.g., rectangular) cross section (along its longitudinal axis LA and/or orthogonal to the longitudinal axis LA). In some aspects, the cross-sectional area of the tissue traction device 110 is larger than the delivery device 120. In some aspects, the distalmost end of the elongate element which forms the delivery device 120 and the tissue traction device 110 is formed with a sharp pointed distal end, such as to facilitate secure engagement with (as well as to facilitate penetration of) the target tissue, such as described above. In some aspects, the exterior of the tissue traction device 110 is smooth to facilitate smooth and controlled insertion into tissue without catching on or tearing the tissue or creating an opening larger than the cross-sectional area of the tissue traction device 110.

In some aspects, a weakened area 115 may be formed between the tissue traction device 110 and the delivery device 120 (e.g., separating the two regions of a common elongate element forming the tissue traction device 110 and the delivery device 120) to facilitate separation of the tissue traction device 110 from the delivery device 120 for deployment within a patient. In some aspects, the weakened area 115 is formed to facilitate specifically intentional separation, without being so weak as to separate during navigation and/or delivery to a deployment site (even along curved or tortuous pathways within the patient's body). For instance, the weakened area 115 may be a frangible region, such as having a thinner cross-section than either or both of the tissue traction device 110 and the delivery device 120. In the example of an embodiment illustrated in FIG. 2, and in greater detail in the detail view of FIG. 2A, the weakened area 115 may be formed by a nick, notch, partial cut, or other type of breakaway formation or structure in the material forming the delivery device 120 and the tissue traction device 110. Once the tissue traction device 110 portion of the elongate element forming the tissue traction device 110 and the delivery device 120 is engaged with tissue (e.g., securely deployed with respect to tissue, such as, without limitation, implanted in tissue, and thus held substantially stationary in place with respect to tissue), the proximal portion of the elongate element, forming the delivery device 120, may be moved with respect to the distal portion of the elongate element, forming the tissue traction device 110, to separate the two portions of the elongate element. For instance, the delivery device 120 may be twisted or torqued with respect to the tissue traction device 110 when the tissue traction device 110 is held substantially stationary with respect to tissue to cause the weakened area 115 to separate (e.g., break apart) the tissue traction device 110 from the delivery device 120.

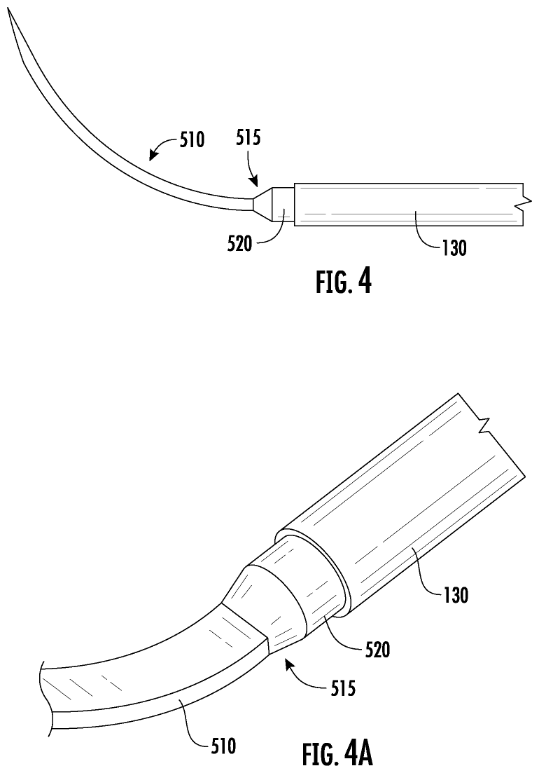

Instead of being monolithic, a tissue traction device and delivery device formed in accordance with various principles of the present disclosure and usable in a tissue traction system 100 as illustrated in FIG. 1, may be formed from separate components which are operably coupled together, such as for delivery of the tissue traction device to a target tissue for deployment. An example of an embodiment of a tissue traction device 510 and delivery device 520 which are formed separately yet operably coupled together, in accordance with various principles of the present disclosure, is illustrated in FIG. 4 and the detail view of FIG. 4A. may be formed from separate elements which are joined together along a frangible joint 515. As may be appreciated, in such example of an embodiment, the tissue traction device 510 may be formed from a shape memory material shape set into a curved configuration (such as any of the shapes described above), such as to apply traction to tissue, whereas the delivery device 520 may be formed from a different material which need not be a shape memory material. The tissue traction device 510 is illustrated in FIG. 4 and FIG. 4A distally extended out of a delivery sheath 130 to an unconstrained configuration. As with the tissue traction device 110 illustrated in FIG. 1, the tissue traction device 510 may be substantially straight when constrained within the lumen 131 of the delivery sheath 130. Once the tissue traction device 510 has been delivered to a target tissue (and distally extended out of the delivery sheath 130) and engaged with respect to the target tissue, the delivery device 520 may be moved (e.g., twisted) with respect to the secured/stabilized tissue traction device 510 to cause the frangible joint 515 to separate, break, etc., to separate the tissue traction device 510 from the delivery device 520 to deploy the tissue traction device 510 within patient's body.

An example of an embodiment of a tissue traction system 100 formed in accordance with various principles of the present disclosure in use during a procedure is schematically illustrated in FIGS. 5A-5D. The tissue traction device 110, delivery device 120, and delivery sheath 130 are illustrated as inserted through the working channel 141 of a medical scope 140 (e.g., endoscope, gastroscope, colonoscope, etc.) inserted into a patient's body to a target tissue T such as to perform an ESD procedure. As described above, the distal portion of the elongate element forming the delivery device 120 is shape set to define a tissue traction device 110 such as with a curved or crescent shaped. The tissue traction device 110 is generally in a straight configuration when stored inside the delivery sheath 130 and delivered to the target tissue T, such as illustrated in FIG. 5A. The pointed sharp distal tip 110t of the tissue traction device 110 can be pushed or distally advanced out of the delivery sheath 130 into the target tissue T (e.g., the mucosal region or layer of the target tissue T) to be engaged with and deployed with respect to tissue, as illustrated in FIG. 5B. In some aspects, the delivery sheath 130 may be proximally retracted to expose the tissue traction device 110 in contrast with distally advancing the tissue traction device 110 out of the lumen 131 of the delivery sheath 130, or a combination of distal advancement of the tissue traction device 110 and proximal retraction of the delivery sheath 130 may be performed. As may be appreciated, and as noted above, the target tissue T constrains the tissue traction device 110 against taking on its relaxed curved shape (the shape-memory set shape of the tissue traction device 110), such as illustrated in FIG. 5B. The delivery device 120, which is formed along the proximal portion of the elongate element which also forms the tissue traction device 110, can be twisted to detach the tissue traction device 110 from the delivery device 120 at the weakened area 115. The delivery sheath 130 and delivery device 120 are then removed (e.g., proximally withdrawn) from the working channel 141 of the medical scope 140, and a medical instrument 150 (e.g., knife, electrocautery device, scissors, etc.) is advanced distally through the working channel 141 to the target tissue T to cut tissue (e.g., submucosal tissue below or surrounding the target tissue T), as illustrated in FIG. 5C As the target tissue T is cut away from surrounding tissue and begins to form a tissue flap, as illustrated in FIG. 5D, the constraining force of the surrounding tissue is released from the cut flap of tissue (in which the tissue traction device 110 has been deployed/implanted), and thus also from the tissue traction device 110 implanted/embedded therein. As illustrated in FIG. 5D, the tissue traction device 110 can thus take on its curved shape, such as by virtue of the shape-memory retention property of the material forming the tissue traction device 110.

It will be appreciated that the various configurations (curvature, thickness, length, relative proportions, etc.) of a tissue traction device to be deployed with respect to a target tissue may be selected by the medical professional such as based on the target tissue and/or surrounding tissue and/or the procedure to be performed. In some aspects, various configurations of tissue traction device 110 are available for use by a medical professional during a procedure. It will further be appreciated that a single tissue traction device or multiple tissue traction devices may be used in a given procedure. Use and deployment of more than one tissue traction device 110 formed in accordance with various principles of the present disclosure is schematically illustrated in FIG. 6A and FIG. 6B. For example, once a first tissue traction device 110 is delivered by a tissue traction system 100 formed in accordance with various principles of the present disclosure, such as illustrated in FIG. 6A (such as described above with reference to FIG. 5A and FIG. 5B, reference being made to the above description thereof for the sake of brevity and without intent to limit), a second tissue traction device 110 may be deployed alongside the first tissue traction device 110, such as illustrated in FIG. 6B. The deployment of one or more additional tissue traction devices 110 (such as the deployment of a second tissue traction device 110 as illustrated in FIG. 6B) may be performed in a manner as described above with reference to FIG. 5A and FIG. 5B, reference being made to the above description thereof for the sake of brevity and without intent to limit. It will be appreciated that selection of the one or more tissue traction devices and placement thereof relative to the target tissue T and to one another may be determined by the medical professional to provide the desired lift to the target tissue T to aid in the performance of a procedure (such as, without limitation, resection) with respect to the target tissue T. It will be appreciated that if multiple tissue traction devices are used, the tissue traction devices need not all be identical, and may be selected based on the target tissue and/or surrounding tissue and/or the procedure to be performed and/or other tissue traction devices being used (and placement thereof).

As may be appreciated, a tissue traction system of the present disclosure allows for a tissue traction device to be delivered by a delivery device without the need for a separate actuation element or other element or device to separate or deploy the tissue traction device from the delivery device. As such, the delivery and deployment of a tissue traction device is simplified, with fewer instruments and steps than required by prior art devices and systems and method. Furthermore, the tissue traction device, once deployed, does not require further instruments to be actuated or otherwise to lift the target tissue away from the instruments being used to perform the procedure. As such, the procedure is not complicated by additional instruments which require manipulation by the medical professional (in addition to the instruments which must be manipulated to perform the procedure) and which may occupy the limited anatomical area at which the procedure is being performed.

It will be appreciated that devices and systems of the present disclosure may be used for treating various anatomical tissue and/or anatomical systems, such as gastrointestinal, abdominal, digestive, urinary, reproductive, respiratory, cardiovascular, circulatory, etc. It will be appreciated that reference to a body passage includes naturally-existing passages (e.g., the colon) as well as medically-created passages (e.g., a passage created with the use of a medical instrument, and not existing without medical intervention) or otherwise. It will further be appreciated that the medical devices and systems described herein may be navigated within and through various body lumens, or other lumens, including, for example, catheters, ureteroscopes, bronchoscopes, colonoscopes, duodenoscopes, arthroscopes, cystoscopes, hysteroscopes, and the like. The disclosed medical devices and systems may also be inserted via different access points and approaches, e.g., percutaneously, endoscopically, laparoscopically, or combinations thereof.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. In particular, it will be clear to those skilled in the art that principles of the present disclosure may be embodied in other forms, structures, arrangements, proportions, and with other elements, materials, and components, without departing from the concept, spirit, or scope, or characteristics thereof. Other examples of manners of implementing the disclosed principles will occur to a person of ordinary skill in the art upon reading this disclosure. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations, and that the various separate features of the present subject matter need not all be present in order to achieve at least some of the desired characteristics and/or benefits of the present subject matter or such individual features. Moreover, elements shown as integrally formed may be constructed of multiple parts or elements shown as multiple parts may be integrally formed, the operation of elements may be reversed or otherwise varied, the size or dimensions of the elements may be varied. It should be apparent to those of ordinary skill in the art that variations and/or modifications, such as of structure, arrangement, proportions, materials, components, and otherwise, can be applied to the disclosed devices, systems, and/or methods, and/or to the sequence of steps of the method described herein without departing from the concept, spirit, and scope of the disclosure. For instance, variations or modifications may be made to adapt devices, systems, or methods of the present disclosure to specific environments and operative requirements without departing from the principles or spirit or scope of the present disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes, modifications, and/or additions apparent to those skilled in the art are deemed to be within the spirit, scope, and concept of the disclosure as defined by the appended claims. Various further benefits of the various aspects, features, components, and structures of tissue traction devices, systems, and methods such as described above, in addition to those discussed above, may be appreciated by those of ordinary skill in the art. The foregoing discussion has broad application and has been presented for purposes of illustration and description and is not intended to limit the disclosure to the form or forms disclosed herein.

The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An implantable tissue traction device configured to be implanted in tissue, said tissue traction device comprising an elongated biocompatible shape memory material defining a distal end and a proximal end of said tissue traction device, wherein:

2. the distal end is formed as a sharp pointed end capable of penetrating into target tissue within a patient's body; and

3. said shape memory material is set into a curved configuration constrained by the target tissue into a substantially straight configuration and releasable into a relaxed curved configuration upon cutting of the target tissue from surrounding tissue.

4. The tissue traction device of claim 1, wherein the proximal end is tapered.

5. The tissue traction device of claim 1, wherein the radius of curvature of said tissue traction device varies along the length thereof.

6. The tissue traction device of claim 1, wherein said tissue traction device has a middle region with a radius of curvature larger than a radius of curvature of either of the proximal end or the distal end.

7. The tissue traction device of claim 1, wherein said tissue traction device has a curved middle region, and the proximal end and the distal end extend obliquely with respect to the middle region.

8. The tissue traction device of claim 1, wherein the tissue traction device has a thickness which varies along the length of the tissue traction device.

9. The tissue traction device of claim 1, wherein the proximal end is separably coupled to a delivery device.

10. The tissue traction device of claim 7, wherein a weakened area is defined between said tissue traction device and the delivery device to facilitate separation of said traction device from the delivery device to deploy said tissue traction device with respect to the target tissue.

11. The tissue traction device of claim 7, wherein said tissue traction device is formed as a distal portion of an elongate element forming the delivery device.

12. The tissue traction device of claim 7, wherein said tissue traction device is separately formed from and operably separably coupled to a distal end of the delivery device.

13. A tissue traction system comprising:

14. a tissue traction device formed of an elongated biocompatible shape memory material defining a distal end and a proximal end of said tissue traction device;

15. a delivery device separably coupled with the proximal end of said tissue traction device; and

16. a delivery sheath defining a lumen through which said tissue traction device and said delivery device are advanceable for delivery to a target tissue within a patient;

17. wherein said tissue traction device is constrained in a constrained substantially straight configuration within the lumen of said delivery sheath and relaxes to a relaxed curved configuration when extended distally out of said delivery sheath.

18. The tissue traction system of claim 11, wherein said tissue traction device and said delivery device are formed from the same monolithic elongate element.

19. The tissue traction system of claim 11, wherein a weakened area is defined between said tissue traction device and said delivery device to facilitate separation and deployment of said tissue traction device with respect to the target tissue.

20. The tissue traction system of claim 13, wherein said tissue traction device and said delivery device are formed of a wire and the weakened area is a cut in the wire.

21. The tissue traction system of claim 11, wherein only the distal portion of the monolithic elongate element is shape set to form said tissue traction device.

22. The tissue traction system of claim 11, wherein said tissue traction device and said delivery device are separately formed and separably coupled together.

23. The tissue traction system of claim 11, wherein said tissue traction device and said delivery device are coupled together via a frangible connection.

24. A method of applying traction to a target tissue, said method comprising:

25. advancing a delivery device to the target tissue, the delivery device having a tissue traction device with a proximal end separably operably coupled to a distal end of the delivery device;

26. advancing a distal end of the tissue traction device into the target tissue, the tissue traction device being formed of a shape memory material shape set into a curved configuration which is constrained by the target tissue into a constrained configuration;

27. separating the proximal end of the tissue traction device from the distal end of the delivery device; and

28. cutting the target tissue with respect to surrounding tissue, thereby releasing the constraining force of the target tissue on the tissue traction device to allow the tissue traction device to return to a relaxed curved configuration lifting the target tissue as it is being cut from surrounding tissue.

29. The method of claim 18, further comprising advancing the tissue traction device and the delivery device to the target tissue within a delivery sheath which constrains the tissue traction device into a constrained configuration.

30. The method of claim 18, further comprising breaking a connection between the tissue traction device and the delivery device to separate the tissue traction device from the delivery device to deploy the tissue traction device with respect to the target tissue.
